(19) United States
(12) Patent Application Publication
(10) Pub. No.: US 2016/0128837 A1
(43) Pub. Date: May 12, 2016

(54) CERAMIC COMPONENTS FOR REPLACING JOINT SURFACES

(71) Applicant: CERAMTEC GMBH, Plochingen (DE)

(72) Inventors: Mateusz Juszczyk, Velden (DE); Alfons Kelnberger, Röthenbach (DE); Christoph Krause, Stuttgart (DE); Heinrich Wecker, Eckental (DE); Frank Ziermann, Berlin (DE)

(21) Appl. No.: 14/899,330

(22) PCT Filed: Jun. 18, 2014
(86) PCT No.: PCT/EP2014/062862

§ 371 Date: Dec. 17, 2015

(30) Foreign Application Priority Data
Jun. 18, 2013 (DE) 10 2013 211 399.4

(51) Int. Cl. A61F 2/30; B28B 11/24; B28B 1/00; B28B 11/12; B28B 1/26; B28B 1/24

(52) U.S. Cl. CPC A61F 2/30756; A61F 2/3094; B28B 1/26; B28B 1/24; B28B 1/001; B28B 1/002; B28B 11/12; B28B 11/243; B28B 1/008; A61F 2002/30011; A61F 2002/30299; A61F 2002/3085; A61F 2002/3092; A61F 2310/00179; A61F 2002/30968

(57) ABSTRACT

Ceramic components for replacing joint surfaces, and methods for production thereof. In particular, the invention relates to ceramic joint surface implants which completely or partially replace natural cartilage.

The invention relates to ceramic components for replacing joint surfaces, and methods for production thereof. In particular, the invention relates to ceramic joint surface implants which completely or partially replace natural cartilage.

Components for partial restoration of joint surfaces are known and are implanted, in particular when irreparable cartilage damage occurs, for example due to arthritis, in the most varied joints in the human body and leads to impairments.

In contrast to total endoprostheses, in this case only a part of the articulation surface is restored, and as a rule the surgery can be performed in a minimally invasive manner.

These partially endoprosthetic implants may be understood as a treatment concept for pain reduction in patients for whom total endoprosthetic surgery would be too risky or would not be suitable because of their active lifestyle. The potential remains for a revision for a total endoprosthesis. Older patients could likewise benefit from a partial endoprosthesis if for example they refuse a total joint replacement because of a higher morbidity or if the surgery appears too dangerous.

Prosthetic components which are known from the prior art for hip, knee, shoulder and small joints are based on metal materials such as titanium or cobalt chromium, wherein these implants function in two parts.

As a rule they have a tribologically stressed part which serves as an articulation surface and an osseointegrating part which grows into the bone tissue and ensures secure anchoring.

The disadvantages of the metal-based solutions are known:
metal abrasion and negative effects on the human organism resulting therefrom,
artefacts in radiological imaging for medical diagnostics,
ageing effects and long-term behavior (fatigue, corrosion, release of metal ions which may have a toxic effect).

An increased risk of infection during the operation has been identified as an additional problem. This applies in particular to metal implants. This risk can be significantly reduced by the use of ceramic, for example a ceramic comprising aluminum oxide and zirconium oxide.

Moreover, so-called osteochondral implants are known which are based on human cartilage and bone tissue. These implants can be implanted into the damaged regions in a minimally invasive manner.

Osteochondral implants have the following disadvantages:
high wear and low strength, because the implant is based on bone and cartilage tissue,
ageing effects and insufficient long-term performance,
high risk of an infection, since human tissue (allograft) serves as a basis for the trabecular structure.

These disadvantages give rise to the objectives of the invention: A joint surface implant as well as a method for production thereof should be provided, which individually or in combination fulfil the following objectives:
After the implantation, the ceramic component should grow together reliably with the bone tissue of the joint and should ensure sufficiently strong anchoring and stability.
After the implantation, the ceramic component together with the rest of the joint surface should constitute a compact unit from the tribological point of view and should be operatively connected securely and permanently to the articulation partner.
The ceramic component should not entail any consequences harmful to health during its residence time in the human body. In particular:
   it should not produce any abrasive particles which are harmful to health;
   in interaction with the articulation partner It should not cause long-term damage to the articulation partner;
   it should not be damaged or destroyed by the biomechanical conditions;
   it should not offer favorable conditions for bacteria which cause infections.

The objective is achieved by a ceramic joint surface implant which comprises a component with at least a first and a second unit, wherein the first unit comprises a dense ceramic and the second unit comprises a ceramic which is porous at least in some regions. The ceramic component or joint surface implant serves for partial restoration of joint surfaces in the human body. The joint surfaces may for example be surfaces from the shoulder, hip, knee and entire foot region.

The implant according to the invention consists of two functional units on the basis of ceramic materials, wherein the first unit is made of a dense ceramic which is optimized for load bearing and furthermore has a tribological surface which is suitable for articulation.

The second unit is, at least in some regions, porous and preferably osseointegrative, i.e., optimized so that it enables the implant to grow into the bone. The porosity of the second unit can either extend over the entire volume of the unit or can also be present only partially, for example on the surface thereof. Furthermore, the porosity can be graded. Thus, it is possible, for example, to design the surface of the second unit to be highly porous, so that a large surface area is provided for growing in of the bone. Towards the interior of the component the porosity can then slowly decrease continuously or step by step.

The ceramic material is preferably an oxide ceramic from the class of aluminum oxides and/or zirconium oxides, for example aluminum oxide with the highest degrees of purity or yttrium-stabilized zirconium oxides or mixed ceramics such as zirconium oxide-reinforced aluminum oxides or aluminum-reinforced zirconium oxides.

Because of their chemical composition, hardness and strength these materials have extremely good tribological characteristics, and during the implantation they are to a large extent damage-tolerant and are more than capable of meeting the biomechanical requirements.

Advantageously, all further developments of these materials are for example extremely damage-tolerant materials such as for example rare earth stabilized dispersoid ceramics consisting of zirconium oxide with fractions of aluminates.

Naturally non-oxidic ceramics, such as for example Si₃N₄-based materials, are also conceivable.

The two functional units are
a tribologically stressed first unit which may comprise a dense ceramic,
an osseointegrative second unit which consists of a ceramic with porous fractions.

The tribologically stressed first unit does not necessarily have to be made of a ceramic material, but may also be a polymerized plastic. Particularly suitable materials are plastics based on polyvinyl alcohols (PVA), which are present at a first time in a viscous liquid state and plastics based upon cross-linking reactions in a second state, which are present in a highly viscous solid state.

In the first state the second osseointegrative unit can be completely or partially infiltrated with the liquid polymer, so that after the cross-linking a tribologically very suitable first unit exists which is connected to an osseointegratively very suitable second unit.

These two units form a compact component and can be firmly connected to one another, in particular by cohesive bonding. Also screwing or some other positive or non-positive connection of the two units is conceivable.

Accordingly, in a method for producing a ceramic joint surface implant, comprising at least a first and a second unit, wherein the first unit comprises a dense ceramic and the second unit comprises a ceramic which is porous at least in some regions, the first unit is connected to the second unit by cohesive bonding, by non-positive or positive engagement.

A connection of the two units by cohesive bonding is particularly preferred. A cohesively bonded connection can be achieved by joint sintering of the two components. The components can be produced in a joint working step or can be manufactured separately. The first and the second unit are brought into contact with one another, preferably physically, as blanks and are jointly sintered.

The tribological surface of the first unit preferably has a curvature which is formed to be complementary to the curvature of the surface of the articulation partner. The curvature is preferably spherical.

According to a quite particularly preferred embodiment of the invention, not only the tribological surface of the first unit but also a surface of the second unit opposite the tribological surface is curved. This curvature may also be preferably spherical. This embodiment of the invention has the advantage that the implant can be easily centered during implantation. It even has a self-centering action when it slides into the optimal position in contact with the articulation partner. Thus, the preferably spherical curvature of the osseointegrative part enables centering in the bone.

A further embodiment of the invention is substantially mushroom-shaped. The first unit of the ceramic joint surface implant is cap-shaped and comprises the tribological surface. The second unit comprises the stem of the mushroom which serves for fastening in the bone.

For fastening of the ceramic joint surface implant in the bone a thread can be provided which can be screwed into the bone. The thread is preferably ceramic and in particular is machined directly out of the implant, i.e., is manufactured integrally. In this case the thread shape is highly geared to the customary design of modem bone screws based on non-ceramic material, although modifications compatible with ceramic are necessary in order for example to avoid tensile or point loading.

Various coatings can be provided for improvement of the osseointegrative action. A coating of titanium which can be applied by sputtering is particularly preferred. This coating on the one hand has a particularly high biocompatibility. Furthermore, the sputtered titanium coating forms a particularly firm and cohesive connection, in particular to oxide ceramic.

The implant can have a biofunctional surface. Bioglass coatings, coatings based on hydroxylapatite or on tricalciumphosphate are particularly preferred. Of course other coatings are possible which stimulate bone growth and are known to the person skilled in the art.

A bioglass composition which has proved particularly successful comprises the compounds SiO₂, CaO, Na₂O and P₂O₅, in particular in a composition consisting of 46.1 mol % SiO₂, 26.9 mol % CaO, 24.4 mol % Na₂O and 2.5 mol % P₂O₅.

The described ceramic joint surface implants are preferably used as cartilage implants which can completely or partially replace the natural cartilage in a joint.

The first and/or the second unit can be formed for example in a conventional dry pressing process, optionally with a subsequent milling process, or by ceramic injection molding (CIM) or low pressure injection molding (LIM).

For the first unit, production is also possible on the basis of a ceramic film which is adapted to the topography of the second unit. With this embodiment anatomically suitable radii can be configured particularly well for the tribologically stressed surface.

Furthermore, it is conceivable for the first unit to be shaped by means of slip casting with a corresponding casting mold and subsequent green machining.

The connection of the two units can also take place by means of an intermediate layer, for example with a ceramic slip based on material of the same type, which connects the two units to one another in the green state (slip jointing). Material of the same type is understood to be a ceramic slip which consists of the same ceramic raw materials as the first and/or the second unit, wherein preferably the composition is also the same in terms of quantities.

According to a particularly preferred embodiment of the invention, for production of the first and the second unit one single (basic) slip is produced which is used for the production of both units. Depending upon the production process, for the second unit for example pore-forming substances can be added to the slip. The slip can also be adapted rheologically to a foaming process. However, the basis of the material is preferably the same as the material for the first unit.

The firm connection of the first and the second unit can then be produced particularly preferably by means of a joint sintering process (co-sintering).

It is also conceivable for the separately manufactured second unit to be dipped in a ceramic slip or for the slip to be applied to it so that after the co-sintering a dense layer is produced which forms the first unit.

Furthermore, it is conceivable for the separately manufactured second unit to be overmolded, in particular as a blank, in a ceramic injection molding process, for example by conventional procedures of the OEM process in the high-pressure range or by conventional low-pressure injection molding processes (LIM). Thus, the first unit can also be constructed on the second unit and after sintering it can be finished mechanically, for example by grinding and polishing.

It is also possible to embed the first prefabricated unit, in particular as a blank, in foam by a foaming process, for example by means of freeze direct foaming on the basis of a suitable slip, and thus to create a porous second unit.

In the first case production methods which are based on direct molding processes are suitable. The direct molding of polymer-based substrate supports, in particular polyurethane foams, is for example suitable here. In this case a polyurethane foam as substrate support is immersed in a slip and the polyurethane foam is then burned out. A primary and a secondary porosity remain. The primary porosity is based on the porous structure of the foam. The secondary porosity is produced in the form of hollow webs in the regions from which the foam has been burned out.

Naturally, foams other than polyurethane can also be used. In this connection it is only important that they can be burned out.

Processes are also conceivable in which the porosities are introduced into correspondingly stabilized slips by other form-foaming means, for example also by stirring in of air bubbles.

A further particularly preferred process for production of such an implant provides for application of a second unit to a blank of the first unit as a slip which contains the pore generator. The pore generators can be mixed with the slip or can be separately applied to the surface for example by blowing on. Consequently pore generators are applied at least to the surface of the slip. They are then burned out, for example during sintering or in another thermal process step.

Also generative manufacturing processes are suitable both for the production of the first unit, but especially naturally also for the porous second unit. The second unit can then be defined with respect to its structure and can be constructed in a targeted manner.

Rapid prototyping processes, in particular processes of stereolithography or fused deposition modelling, are preferred as generative manufacturing processes. By means of this process a substrate support can be defined for a later ceramic coating process and be produced in precisely the required or optimal structure.

However, processes are also possible in which the blank is produced in particular for the second unit by means of a generative manufacturing process, that is to say without the intermediate step of production of a substrate support. In this connection the ceramic inkjet direct printing process or the 3D powder bed printing process is particularly suitable.

The invention is explained in greater detail below with reference to individual examples.

In a preferred embodiment, the implant according to the invention consists of a first unit which is produced on the basis of a dry pressing process and is connected to, in particular co-sintered with, a trabecular second unit produced on the basis of a direct molding process.

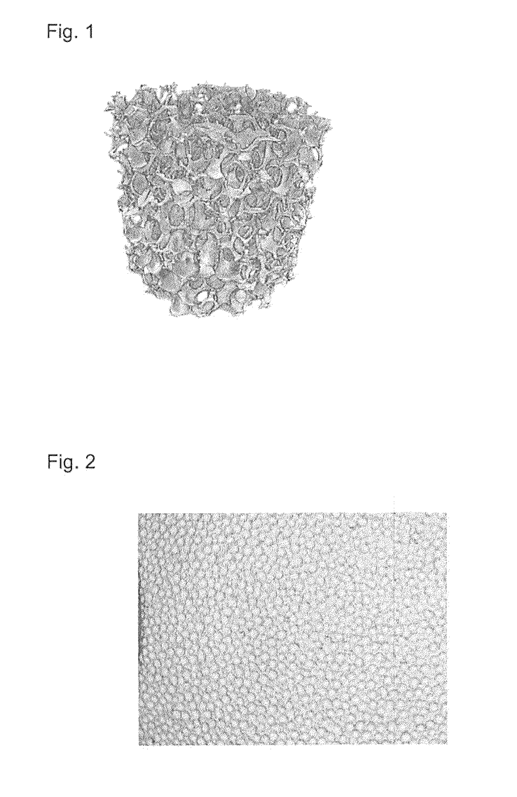

FIG. 1 shows such a trabecular structure produced by direct molding as a second unit. A polyurethane foam has been impregnated with a ceramic slip, the polyurethane substrate support has been burned out and the structure has been sintered.

The result of a further preferred process according to the invention for production of the porous second unit is shown in FIG. 2. Here a ceramic slip has been applied to a blank of the first unit and a pore generator has been applied, in this case blown on, to the surface of the slip. The pore generators were burned out during subsequent sintering.

A plurality of organic substances, but also inorganic substances, are suitable as pore generators. They must be easily crushed and must burn out, preferably substantially without residue, at the most at the sintering temperature of the ceramic. For example, polymer-based beads on the basis of polyvinyl acetate or polyvinyl butyral can be used.

This embodiment of the invention is only superficially porous, the pores are not interconnecting. However, they offer a large and greatly split surface for the growing in of bone tissue. If the primary stability of an implant with such a surface is ensured, the quantity of bone cement necessary can advantageously be reduced or bone cement can even be omitted completely.

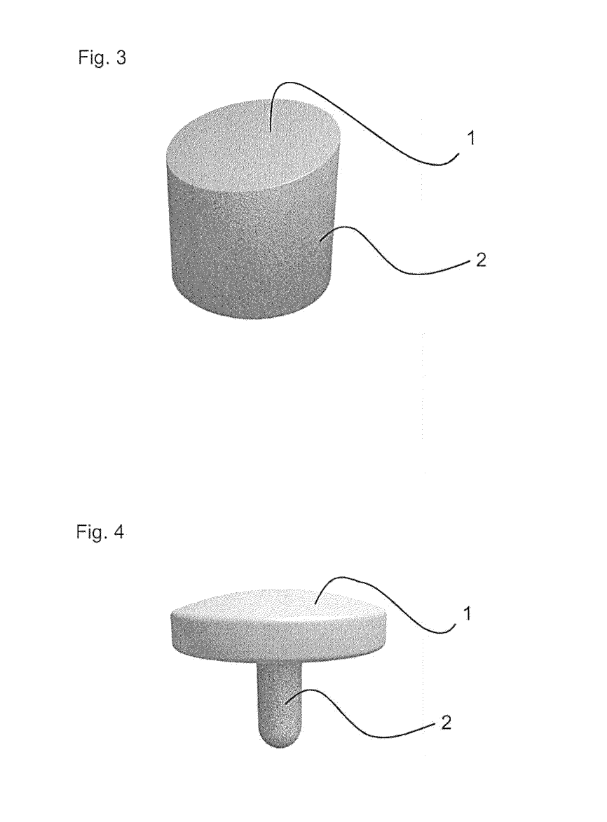

FIG. 3 shows an exemplary embodiment of an implant according to the invention for cartilage replacement in a knee joint. The joint surface implant consists of a ZTA (zirconia toughened alumina) ceramic. The first unit 1 has a tribological surface and the second unit 2 has an osseointegrative surface. The osseointegrative surface of the second unit is produced as described for FIG. 2.

The tribological surface of the first unit 1 has a spherical curvature and is adapted to the anatomy of the joint surface, so that in particular discontinuous transitions between the implant and the cartilage material of the joint surface are avoided.

A further functionally very similar variant is illustrated in FIG. 4. As described for FIG. 2, the second unit is produced by a sintered ceramic slip with burned out pore generators.

The dimensions differ from those of FIG. 3. The tribological surface of the first unit 1 is greatly enlarged by comparison with the second unit 2 which serves for anchoring of the joint surface implant. The implant has a mushroom shape, wherein the cap corresponds to the first unit 1 with the tribological surface and the stem corresponds to the second unit 2 with the osseointegrative characteristics. This may be advantageous for specific indications, for example for extremely osteoporotic bone substance.

In a further embodiment screw-like structures are possible, with a type of screw head, which according to the tribological requirements is correspondingly ground and polished after the sintering, and is equipped with a thread design which is compatible with the ceramic and adapted to the bone tissue, similar to a dental implant.

The osseointegrative coating is sprayed on with a sprayable slip and a pore generator is blown onto the surface.

A further method for promoting the osseointegration is the application of a high-strength titanium sputter coat with a thickness in the region of several micrometers to the surface of the second unit. As a rule, in such processes a chemical intermediate layer forms on a microscopic plane with the atomic constituents of the ceramic, in a ZTA ceramic for zirconium and aluminum, as well as titanium. This layer ensures an extremely, good adhesion of the actual titanium layer, which in turn forms an outstandingly suitable surface for the growth of bone cells. Such layers are known for their osseointegrative characteristics.

In order to increase the osseointegration capacity, the component or the second porous unit can be biofunctionalized by suitable processes and methods. This can take place for example by application of bioglass coatings, inter alia by means of dipping processes, sol-gel processes or electrophoretic deposition processes.

Bioglasses are typically mixtures of different oxides which are applied in an amorphous structure to the ceramic components to be coated. Bioglasses made of SiO₂, CaO, Na₂O and P₂O₅ are particularly preferred.

A quite typical and very efficient variant with a view to activating bone growth is a bioglass with the following composition:
46.1 mol % SiO₂ (silicon dioxide)
26.9 mol % CaO (calcium oxide)
24.4 mol % Na₂O (sodium hyperoxide)
2.5 mol % P₂O₅ (phosphorus pentoxide)

Naturally other bioglass compositions are also conceivable, or also conventional osseointegrative coatings on the basis of hydroxylapatite or tricalciumphosphate (TCP).

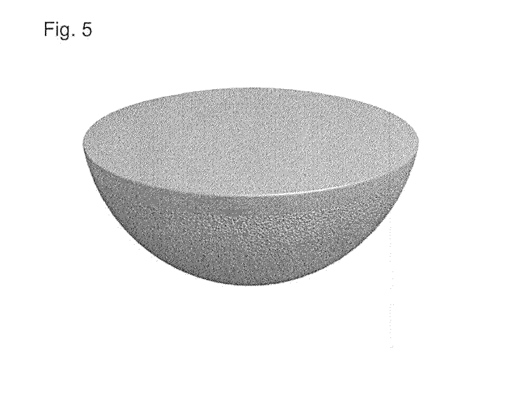

FIG. 5 shows a particularly preferred exemplary embodiment of the invention. The illustrated implant is a self-centering implant. In this case not only the tribological surface of the first unit, but also the osseointegrative surface of the second unit opposite said tribological surface, is curved spherically. This embodiment has the advantage that the curvature of the first tribological unit in the implantation into the bone tissue of the joint to be repaired can be very easily centered and in fact centers itself upon contact with the counter-articulation surface.

The subsequent healing in the bone tissue fixes this optimal position and ensures a durable partial endoprosthesis.

1.-16. (canceled)

17. A ceramic joint surface implant comprising:
a component with at least a first unit and a second unit;
wherein the first unit comprises a tribological surface which is suitable for articulation, and
wherein the second unit comprises a ceramic which is porous at least in some regions.

18. The ceramic joint surface implant according to claim 17, wherein the first unit comprises a dense ceramic.

19. The ceramic joint surface implant according to claim 17, wherein the second unit has a surface which is osseointegrative.

20. The ceramic joint surface implant according to claim 18, wherein the tribological surface has a curvature.

21. The ceramic joint surface implant according to claim 20, wherein a surface of the second unit opposite the tribological surface of the first unit is likewise curved.

22. The ceramic joint surface implant according to claim 17, wherein the implant has a mushroom shape, wherein the cap of the mushroom comprises the tribological surface and the stem of the mushroom serves for fastening in the bone.

23. The ceramic joint surface implant according to claim 17, wherein the implant has a ceramic thread so that the implant can be screwed into the bone.

24. The ceramic joint surface implant according to claim 17, wherein the implant has an osseointegrative coating.

25. The ceramic joint surface implant according to claim 24, wherein the osseointegrative coating comprises at least one member selected from the group consisting of sputtered titanium, a biofunctional surface, a bioglass, hydroxyapatite and tricalciumphosphate.

26. A method for producing a ceramic joint surface implant, wherein the ceramic joint surface implant comprises at least a first and a second unit, wherein the first unit has a tribological surface which is suitable for articulation and the second unit comprises a ceramic which is porous at least in some regions, comprising the step of connecting the first unit to the second unit by cohesive bonding, by non-positive or positive engagement.

27. The method according to claim 26, wherein the first unit comprises a dense ceramic, and the first and the second unit are brought into contact with one another physically as blanks, and are jointly sintered.

28. The method according to claim 26, wherein the first and the second unit as blanks are connected to one another by means of a ceramic slip, wherein the ceramic slip is preferably produced from the same ceramic raw materials.

29. The method according to claim 26, wherein the first and/or the second unit are formed by slip casting in a casting mold and are then green machined, or wherein that the first and/or the second unit are formed by a dry pressing process, optionally with a subsequent milling process.

30. The method according to claim 26, wherein the first and/or the second unit are formed by a ceramic injection molding process, in particular by ceramic injection molding or low pressure injection molding.

31. The method according to claim 26, wherein for the first unit a ceramic film is produced which is adapted to a topography of the second unit, wherein anatomically adapted radii are produced for a tribologically stressed surface of the first unit.

32. The method according to claim 26, wherein a blank for the second unit is produced by means of a generative manufacturing process, in particular by ceramic inkjet direct printing or by 3D powder bed printing.

33. The method according to claim 26, wherein the blank of the second unit is produced from a foamed slip, wherein the foamed slip is produced in particular by means of freeze direct foaming, in particular by embedding the blank of the first unit in foam, or by introduction of air bubbles into a stabilized viscous slip, for example by stirring in or by foam-generating means.

34. The method according to claim 26, wherein the second unit is applied as a slip to a blank of the first unit and pore generators are applied at least to the surface of the slip, wherein the pore generators are burned out during subsequent sintering.
